(19) United States
(12) Patent Application Publication
(10) Pub. No.: US 2008/0213410 A1
(43) Pub. Date: Sep. 4, 2008

(54) ANTI-HUMAN PAPILLOMAS VIRUS TOPICAL COMPOSITION

(76) Inventor: Michel Serrano, Montreal (CA)

Correspondence Address: BERESKIN AND PARR, 40 KING STREET WEST, BOX 401, TORONTO, ON, M5H 3Y2, CA

(21) Appl. No.: 12/027,558

(22) Filed: Feb. 7, 2008

Related U.S. Application Data
(60) Provisional application No. 60/888,863, filed on Feb. 8, 2007.

(51) Int. Cl. A61K 36/8962; A61P 31/12

(52) U.S. Cl. 424/754

(57) ABSTRACT

The present invention relates to a topical composition for reducing lesion and inflammation of human papillomavirus (HPV) on the skin and mucous membranes comprising an effective amount of at least one component from one of ground garlic, garlic powder, garlic oil, minced garlic, and greater celandine, a terpene-, phenol- or polyphenol-containing agent, and a sulfurated compound in association with a pharmaceutically acceptable carrier to form a homogenous topical composition, and method of treatment thereof.

### BACKGROUND OF THE INVENTION

(a) Field of the Invention

The present invention relates to a topical composition for reducing lesion and inflammation of human papillomavirus (HPV) on the skin and mucous membranes comprising an effective amount of at least one component from one of ground garlic, garlic powder, garlic oil, minced garlic, and greater celandine, a terpene-, phenol- or polyphenol-containing agent, and a sulfurated compound in association with a pharmaceutically acceptable carrier to form a homogenous topical composition, and method of treatment thereof.

(b) Description of Prior Art

Genital warts are known as one of the most common sexually transmitted diseases. 1% of the sexually active people (between age 15 and 45) have genital warts and 40% of adults carry the virus called HPV (human papillomavirus) that causes genital warts. Some 25 million Americans are thought to have HPV. There are more than 60 types of virus, some infecting the skin and causing common warts, and about one-third of the HPV types may be spread sexually and cause genital warts.

No treatment for genital warts is completely effective because it relies on destroying the skin infected by the virus. Surgery by laser, electrocautery and cryogenic methods (liquid nitrogen) are obviously the most efficient ways to remove such infected skin. Unfortunately, it can't be applied to all cases.

Podophyllum resin (extract from May apple and mandrake root) is used as a caustic for warts. Its use is forbidden for pregnant patient. A milder form of podophyllum, and probably the most employed medicine for genital warts, is Condylox® (also known as podofilox). It is recommended for the topical treatment of anogenital warts (external genital and perianal warts) but not for the mucous membrane warts.

Even with a certain success as topic treatment for warts including the genital ones, the antimitotic nature if these compounds is a real drawback with which the users have to deal with. The resulting toxicity limits its use to non-pregnant people, non breast-feeding mothers and for cutaneous treatment only. Indeed, serious following of the recommended dosage is important to avoid carcinogenesis, mutagenesis and impairment of fertility. In vivo tests showed that podofilox should be considered as a potential clastogen (induces disruption and breakage of chromosomes). Furthermore, it is highly recommended to limit the length of the treatment (3 days of application then 4 days of non-application for 4 weeks) to decrease the risk of local adverse reactions of and systemic reactions. Finally, the patient has to be careful during the application, only treating the wart itself and minimizing contact with the non-infected surrounding area.

Imiquimod (sold commercially by 3M Pharmaceuticals under Aldara® as a cream), is an antiviral that does not affect the healthy skin contrary to podophyllum and derivatives.

Other treatments with 5-fluorouracil, bi and trichloroacetic acids and interferon inducers cause irritation of the skin and requires weeks of treatment. An alternative treatment (Condysil®) is based on salicylic acid. Even the fact that this product is fully FDA approved, its efficiency is lower that the other commercial products. Topical applications of vitamin A, lomatium (Lomatium dissectum) isolate, or tea tree oil (Melaleuca altemifolia) helps resolve warts and prevent recurrence of outbreaks. There are other substances which are useful to sustain the healing during the recovering of treatment with antivirals such as vitamin C, aloe, zinc and licorice.

Many types of antiviral medicines have been used to relieve patients from HPV related lesion and inflammation but none of them can really heal the disease itself. Most genital warts are superficial and are treated with ointments with some success but the genital warts are known to have a rate of recurrence. Moreover, none of the know treatment can reach deep warts in the flesh. Therefore, the person infected by this type of wart is without means (with the exception of surgery). It would be highly desirable to be provided with a medicine consisting in a topical, non-irritating formulation, having no or minimal side-effects, that could be applied without restrictive length of application, wouldn't affect non-infected skin, could heal surface and deep flesh warts and prevent the recurrence of outbreaks.

### SUMMARY OF THE INVENTION

In accordance with the present invention there is provided a topical composition for reducing lesion and inflammation of human papillomavirus (HPV) on the skin and mucous membranes comprising an effective amount of at least one components from one of ground garlic, garlic powder, garlic oil minced garlic, and greater celandine, a terpene-, phenol- or polyphenol-containing agent, and a sulfurated compound in association with a pharmaceutically acceptable carrier to form a homogenous topical composition.

In accordance with the present invention, the topical composition further comprises an effective amount of a terpene-, phenol- or polyphenol-containing agent that is at least one essential oil selected from thuja oil, eucalyptus oil, birch oil, Atlantic cedar oil, red thyme oil, oregano oil, tarragon oil, red cedar wood oil, and basil oil eucalyptus oil, birch oil, Atlantic cedar oil, and combination thereof.

In accordance with the present invention, the topical composition further comprising an effective amount of a terpene-, phenol- or polyphenol-containing agent that is capsaicin or cayenne pepper

In accordance with another embodiment of the present invention, there is provided a method for producing said topical composition, said method comprising the steps of grinding garlic cloves in a coffee mill, blending the grinded garlic cloves with 12 drops of thuja oil and pharmaceutically effective amounts of capsaicin and 1000 mg of methyl-sulphone-methane, and/or methylsulfonylfluoride and admixing the combination in a pharmaceutically acceptable carrier.

In accordance with another embodiment of the method of production of the composition, the method further comprises the step of blending 12 drops of eucalyptus oil and/or birch oil and/or Atlantic cedar oil in admixture with the pharmaceutically acceptable carrier.

In accordance with still another embodiment of the method of production of the composition, the method further comprises the step of blending the ingredients in an aqueous solution before admixing the composition in a pharmaceutically acceptable carrier.

In accordance with still another embodiment of the method of production of the composition, the pharmaceutically acceptable carrier of the topical composition consists in a lotion or an ointment.

In accordance with another embodiment of the present invention, there is provided a method for the treatment of HPV related infections and inflammations of skin and mucous membranes comprising administering topically a sufficient amount of the topical composition to the skin or mucous membrane of a patient.

In accordance with another embodiment of the method of treatment, the infections and inflammations comprise any acute or chronic genital wart.

In accordance with still another embodiment of the method of treatment, the topical administration is effected by swiping the surface of the skin or mucous membrane to treat with a swab containing the composition in the case of an acute wart.

In accordance with still another embodiment of the method of treatment, the topical administration is transdermal in the case of a chronic wart, which consists of inoculating the composition under the skin or mucous membrane of the subject with a microlet or applying to the skin of the subject a transdermal patch diffusing the composition trough the skin or mucous membrane over an extended period of time.

In accordance with another embodiment of the present invention, there is provided the use of a sufficient amount of the topical composition for the reduction of HPV related infections and inflammations of skin and mucous membranes comprising topically administering a sufficient amount of the topical composition to the skin or mucous membrane of a patient.

More precisely, the lesion may be a wart, chronic or acute, or of genital origin.

In accordance with still another embodiment of the use of the topical composition for the treatment, the topical administration is effected by swiping the surface of the skin or mucous membrane to treat with a swab containing the composition in the case of an acute wart.

In accordance with still another embodiment of the use of the topical composition for the treatment, the topical administration is transdermal in the case of a chronic wart, which consists of inoculating the composition under the skin or mucous membrane of the subject with a microlet or applying to the skin of the subject a transdermal patch diffusing the composition trough the skin or mucous membrane over an extended period of time.

For the purpose of the present invention the following terms are defined below.

The term “microlet” consists in an instrument intended to inoculate a live subject by inserting a dose of the composition to administer under the skin. This is effected through quick piercing of the skin by a mechanically activated needle containing the dose to administer and liberating it under the skin.

The expression “pharmaceutically suitable carrier” is intended to mean a formulation that presents certain benefits in terms of diffusion and release of the withheld ingredients in relation with the method of administration, the subject it is intended to and the storing conditions.

The expression “a terpene-, phenol- or polyphenol-containing agent” is intended to mean any composition that contains therein terpenes, phenols or polyphenols, amongst others. Such composition includes, without limitation, essential oils, capsaicin and cayenne pepper.

All references referred herein are hereby incorporated by reference.

### BRIEF DESCRIPTION OF THE DRAWINGS

FIG. 1 consists of an illustration of the genome of the HPV, with a schematization of the size and arrangement of the main genes of the virus.

FIG. 2 illustrates the mechanism of entry of the viral genome into the nucleus of epithelial cells and the related translation in mRNAs that are coding for the viral proteins.

### DETAILED DESCRIPTION OF THE INVENTION

In accordance with the present invention, there is provided a topical composition for reducing lesion and inflammation of the human papillomavirus (HPV) on the skin and mucous membranes comprising an effective amount of pure grinded garlic, thuja oil, capsaicin and methyl-sulphone-methane admixed in a pharmaceutically acceptable carrier, said composition further comprising an effective amount of eucalyptus oil, birch oil, Atlantic cedar oil, red thyme oil, oregano oil, tarragon oil, red cedar wood oil, basil oil, sodium acid carbonate and an aqueous solution in a preferred embodiment.

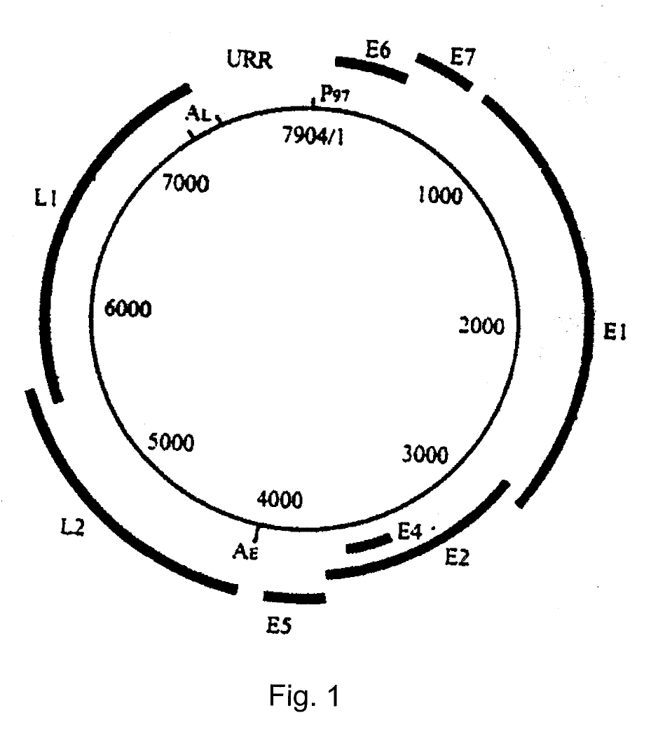

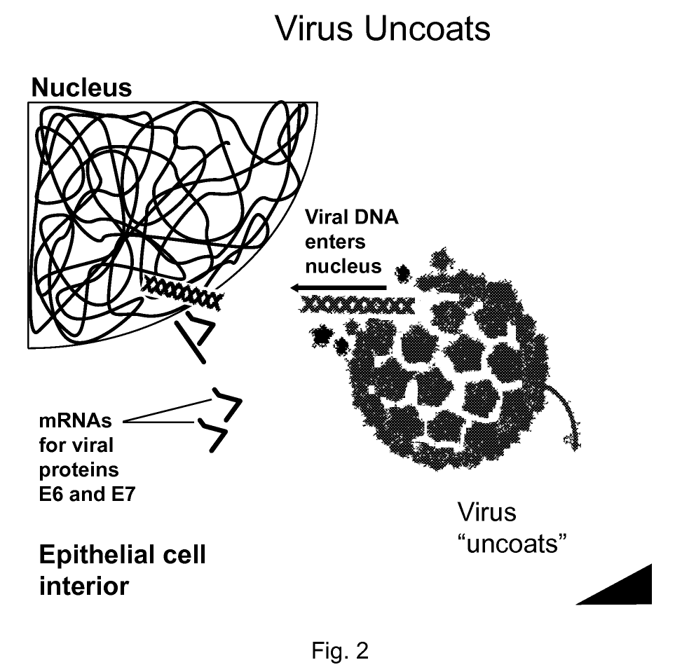

The HPV is responsible for the development of cutaneous and sub-cutaneous wart-like infections and inflammations of the skin, most frequently located in the genital region. There are more than 100 different genotypes for the virus: in most cases of infections, the virus disappears by itself, but in other cases, it will cause various manifestations on the skin and mucous membranes and can even lead to the development of certain types of cancers when left untreated, such as cervical cancer. The virus will integrate the host through interaction of its structural proteins with the cellular target. Endocytosis allows integration of viral particles into the cell followed by incorporation of the new genetic material into the host's DNA. (FIG. 2) Thereafter, HPV is able to replicate its enzymes and produce the material essential to its survival. FIG. 1 consists of a representation of the genome of the HPV: as is depicted, the virus has two different coding regions. The region “E” encodes for genes that are specific to the cellular replication, transcription and transformation of the viral proteins. The region “L” encodes for genes of the viral structural proteins, which are the proteins of the capsid. One to 6 months after the infection, which usually happens through sexual contact between non-infected and infected partners, the incubation period reaches an end and the virus manifests itself. Indeed, various wart-like lesions will appear on infected subjects, specifically in the genitalia region. Rarely isolated, they will emerge as humid, pinkish soft tumefactions that have high transmissible potential. Usually, the HPV related infections of the skin and of the mucous membranes are destructed by doctors through the use of liquid nitrogen, podophyllum and trichloroacetic acid among others. However, the HPV being deeply integrated in the genomic material of the host, treatment of the superficial manifestations of the virus won't destruct it, therefore allowing reappearance of the lesions. Moreover, the E6 and E7 proteins (FIG. 1) of the HPV are able to interfere with cellular cycle regulation. Indeed, these viral proteins link themselves with the hosts' p53 and pRB proteins, which are the “tumor-suppressor” proteins. Such linkage entails inactivation of the apoptosis mechanism of the host's cells, normally leading to a cell's suicide whenever its DNA's repairing system is not working. Therefore, DNA's lesions won't be regularly eliminated, highly increasing the host's risks of developing a cancer.

The topical composition of the present invention proposes a sensible and effective treatment of the HPV related infections. It comprises four principal components in admixture in a pharmaceutically acceptable carrier. The first ingredient is pure grinded garlic. Garlic has been known for centuries as having powerful antiviral activities. Many medicines were developed through the years with this one-of-a-kind vegetable. Indeed, several examples of medical treatment, from cold to anti-bacterial drugs, are found in the folkloric and scientific literature. The main active ingredient of garlic is sulfur, which is highly air-sensitive: use of fresh garlic is therefore essential in order to get the highest possible activity. The sulfur found in garlic is present through the cysteine amino acid. Cysteine is broken down by an interaction with cysteindesulfhydrase, a bacterium present at the surface of the skin, and therefore produces sulfured-hydrogen (H₂S), a very corrosive agent. The second ingredient of the composition is a terpene-, phenol- or polyphenol-containing agent, such as an essential oil, which provides astringent, stimulant (nerves, uterus and heart muscles) and tonic properties to the composition. Essential oils such as thuja oil, eucalyptus oil, birch oil, Atlantic cedar oil, red thyme oil, oregano oil, tarragon oil, red cedar wood oil, and basil oil can be used in the composition. The terpenes are water-soluble and liposoluble molecules: they are easily mixed with either water-based solutions or oil-based solutions. A phenol or polyphenol, such as capsaicin, is the third important ingredient: it is known to provide temporary relief of minor aches and pains. Finally, a sulfurated compound, such as methyl-sulphone-methane or methylsulfonylfluoride, increases the total sulfur amount of the composition, and the related sulfured-hydrogen production, which is the responsible for the anti-HPV effects. Moreover, an aqueous solution, to solubilize the ingredients, and sodium acid carbonate, to plasticize the composition, can be added to the blend.

The topical composition is produced through a series of steps. First, fresh garlic cloves are grinded with a coffee mill to produce a total of 4 to 8 g of the ingredient. The grinded garlic cloves are then combined with 12 drops of thuja oil (0.8 ml or 0.84 mg), an effective amount of capsaicin, and 1000 mg of methyl-sulphone-methane. These ingredients are then incorporated in an admixture in a pharmaceutically acceptable carrier, such as a lotion or an ointment. The carrier is chosen according to its ability to penetrate the skin, in order to reach the warts that are deep in the flesh as well as those that are superficial. The four mentioned ingredients are the essential ones: however, as we mentioned, certain other components can be added. These other components will be integrated, alone or in combination, in effective amounts in the composition. Eucalyptus oil, birch oil and Atlantic cedar oil are added to the blend in an amount of 12 drops (0.8 ml or 0.84 mg), in a preferred embodiment. The aqueous solution, to solubilize the ingredients is added in pharmaceutically acceptable amounts. The sodium acid carbonate is inserted in a quantity equivalent to two teaspoons.

The topical composition is can also be produced through another series of steps. First, 540 mg of garlic powder is combined with 12 drops of thuja oil (0.8 ml or 0.84 mg), an effective amount of capsaicin, and 180 mg of methylsulfonylfluoride. These ingredients are then incorporated in an admixture in 15 g of a pharmaceutically acceptable carrier, such as a lotion or an ointment. The carrier is chosen according to its ability to penetrate the skin, in order to reach the warts that are deep in the flesh as well as those that are superficial. The four mentioned ingredients are the essential ones: however, as we mentioned, certain other components can be added. These other components will be integrated, alone or in combination, in effective amounts in the composition. Eucalyptus oil, birch oil, Atlantic cedar oil, red thyme oil, oregano oil, tarragon oil, red cedar wood oil, and basil oil are added to the blend in an amount of 12 drops (0.8 ml or 0.84 mg), in a preferred embodiment. The aqueous solution, to solubilize the ingredients is added in pharmaceutically acceptable amounts.

The composition, to be applied topically or transdermally to the skin and mucous membranes, is of high interest because it triggers little or no side-effects, very little irritation, no restrictive length of application, no impact on non-infected skin, can heal surface and/or deep flesh warts and prevent the recurrence of outbreaks. The treatment consists in topically administering the composition to the HPV infected skin and mucous membranes of patients. Such physiological regions consists of, in particular, the skin of the penis, vulva (area outside the vagina), anus, scrotum, groin, or thigh and in the linings of the vagina, cervix, or rectum presenting the virus related warts. In vivo results, on a total of 90 patients, demonstrated that the recurrent application on and/or through the skin constitutes a safe and effective method for the treatment genital warts. Two types of warts can be found in patients: acute or chronic warts. The first kind, the acute wart, can be treated by simply swiping the surface of the skin or mucous membrane with a swab, previously impregnated with the composition, and gently rubbing the skin in order to allow penetration of the composition in its deepest layers. The second kind is the chronic wart, which requires a more invasive type of treatment. Indeed, the treatment is either effected by applying to the skin or mucous surface to treat a transdermal patch, constantly and regularly diffusing the composition through the area it covers, or by using a microlet, which uses a needle rapidly piercing the skin and injecting the composition deeply in the flesh.

The present invention will be more readily understood by referring to the following examples, which are given to illustrate the invention rather than to limit its scope.

### EXAMPLE 1

Preparation of the topical composition: preparation of 4 to 8 g of shredded garlic cloves through use of a coffee mill. They are subsequently incorporated in an aqueous solution with 12 drops of thuja oil (0.8 ml or 0.84 mg), an effective amount of capsaicin and 1000 mg of methyl-sulphone-methane. In a preferred embodiment of the invention, 12 drops of birch oil (0.8 ml or 0.84 mg), 12 drops of eucalyptus oil (0.8 ml or 0.84 mg) and 12 drops of Atlantic cedar oil (0.8 ml or 0.84 mg) are added in the composition. Two tablespoons of sodium acid carbonate are also added in a preferred embodiment to increase the plasticity of the composition and allow easier administration on the skin and mucous membranes.

Posology: Application of the topical composition is effected 1 time per week through swiping of the region with a swab containing the composition in the case of an acute wart or through the use of a transdermal patch or microlet in the case of a chronic wart.

Results: Table 1 comprises the results of a 15 months treatment with the composition of the present invention on a total of 90 infected patients. Table 2 comprises the results of a 15 months treatment with liquid nitrogen on a total of 90 infected patients. The compiled data consists of the number of recurrent warts that had still developed after a 15 months treatment with either the cream of the present invention or with liquid nitrogen. These records represent direct evidence that the composition of the present invention is effective, the number of warts that had still reappeared after a 15 months long treatment of the intermittent lesion and inflammation being less important with the cream than with the liquid nitrogen. Indeed, 67% of patients treated with the composition of the present invention presented a low degree of infections after the 15 months treatment (1 to 2 recurrent warts) whereas only 13% of the patients treated with the liquid nitrogen presented similar results.

TABLE 1
Results of a 15 months treatment with the composition of the
present invention
Number of recurrent warts after a 15
months treatment on 90 patients

| Number of warts | Number of patients |
|---|---|
| 6 to 10 | 2 |
| 3 to 5 | 28 |
| 1 to 2 | 60 |

TABLE 2
Results of a 15 months treatment with liquid nitrogen
Number of recurrent warts after a 15
months treatment on 90 patients

| Number of warts | Number of patients |
|---|---|
| 6 to 10 | 29 |
| 3 to 5 | 49 |
| 1 to 2 | 12 |

### EXAMPLE 2

Preparation of the topical composition: 540 mg of garlic powder is combined with 12 drops of thuja oil (0.8 ml or 0.84 mg), an effective amount of capsaicin, and 180 mg of methylsulfonylfluoride. These ingredients are then incorporated in an admixture in 15 g of a cream. In a preferred embodiment of the invention, eucalyptus oil, birch oil, Atlantic cedar oil, red thyme oil, oregano oil, tarragon oil, red cedar wood oil, and basil oil are added to the blend in an amount of 12 drops (0.8 ml or 0.84 mg).

Posology: Application of the topical composition is effected as in Example 1.

Results: Table 3 comprises the results before an 8 weeks treatment with the composition of the present invention or a control composition (lotion only) on a total of 17 infected patients with pubic warts. Table 4 comprise the results after an 8 weeks treatment with the composition of the present invention or a control composition (lotion only) on a total of 17 infected patients with pubic warts. The compiled data consists of the number of recurrent warts that had still developed after an 8 weeks treatment with either the cream of the present invention or the control composition. These records represent direct evidence that the composition of the present invention is effective, the number of warts still present after an 8 weeks long treatment of the intermittent lesion and inflammation being less important with the cream of the composition of the present invention than with the control composition. Indeed, 89% of patients treated with the composition of the present invention presented a lower degree of infections after the 8 weeks treatment (1 to 3 recurrent warts) whereas none of the patients treated with the control composition presented similar results. Furthermore, statistical analysis of the results with a Student's t-test confirms the significance of this effect (p≦0.01).

TABLE 3
Results before an 8 weeks treatment with the composition of the
present invention

| | |
|---|---|
| Anti-VPH | Control |
| composition | composition |

| Subject | Wart | Subject | Wart |
|---|---|---|---|
| 1 | 4 | 1 | 4 |
| 2 | 5 | 2 | 6 |
| 3 | 3 | 3 | 6 |
| 4 | 2 | 4 | 5 |
| 5 | 5 | 5 | 5 |
| 6 | 4 | 6 | 4 |
| 7 | 4 | 7 | 4 |
| 8 | 4 | 8 | 4 |
| 9 | 5 | | |

| | |
|---|---|
| Average = 4 | Average = 4.75 |

TABLE 4
Results of an 8 weeks treatment with the composition of the present
invention

| | |
|---|---|
| Anti-VPH | Control |
| composition | composition |

| Subject | Wart | Subject | Wart |
|---|---|---|---|
| 1 | 2 | 1 | 4 |
| 2 | 1 | 2 | 5 |
| 3 | 2 | 3 | 4 |
| 4 | 3 | 4 | 6 |
| 5 | 2 | 5 | 4 |
| 6 | 1 | 6 | 4 |
| 7 | 1 | 7 | 5 |
| 8 | 2 | 8 | 4 |
| 9 | 3 | | |

| | |
|---|---|
| average = 1.9 | average = 4.5 |

### EXAMPLE 3

Preparation of the topical composition: 540 mg of garlic powder is combined with 12 drops of thuja oil (0.8 ml or 0.84 mg), an effective amount of capsaicin, and 180 mg of methylsulfonylfluoride. These ingredients are then incorporated in an admixture in 15 g of a cream. In a preferred embodiment of the invention, eucalyptus oil, birch oil, Atlantic cedar oil, red thyme oil, oregano oil, tarragon oil, red cedar wood oil, and basil oil are added to the blend in an amount of 12 drops (0.8 ml or 0.84 mg).

Posology: Application of the topical composition is effected 4 time per week before sleep, through swiping of the region under the prepuce with a swab containing the composition.

Results: Table 5 comprises the results of a 6 weeks treatment with the composition of the present invention on a total of 10 infected patients chosen from the group of 90 individuals from Example 1 since they had recurrent clusters of warts specifically under the prepuce. The compiled data consists of the number of recurrent warts that had still developed after a 6 weeks treatment with either the formulation of the cream of the present invention used in Example 1 versus that used in Examples 2 and 3. These records represent direct evidence that the composition of the present invention is effective, as the number of warts still present after a 6 weeks long treatment of the intermittent lesion and inflammation being less important with the cream of the composition of Examples 2 and 3 than with the composition of Example 1. Indeed, 100% of patients treated with the composition of the present invention presented a lower degree of infections after the 6 weeks treatment (1 to 3 recurrent warts). Furthermore, statistical analysis of the results with a Student's t-test confirms the significance of this effect (p<0.01).

TABLE 5
Results of an 8 weeks treatment with the composition of the present
invention

| | Number of clusters | Number of clusters |
|---|---|---|
| | after treatment | after treatment |
| | (Composition | (Composition |
| Subject | of Example 1) | of Example 3) |
| 1 | 3 | 2 |
| 2 | 4 | 2 |
| 3 | 2 | 1 |
| 4 | 3 | 2 |
| 5 | 4 | 2 |
| 6 | 3 | 1 |
| 7 | 5 | 3 |
| 8 | 4 | 2 |
| 9 | 2 | 1 |
| 10 | 2 | 0 |

### EXAMPLE 4

Preparation of the topical composition: 540 mg of garlic powder is combined with 12 drops of thuja oil (0.8 ml or 0.84 mg), an effective amount of capsaicin, and 180 mg of methylsulfonylfluoride. These ingredients are then incorporated in an admixture in 15 g of a cream. In a preferred embodiment of the invention, Eucalyptus oil, birch oil, Atlantic cedar oil, red thyme oil, oregano oil, tarragon oil, red cedar wood oil, and basil oil are added to the blend in an amount of 12 drops (0.8 ml or 0.84 mg).

Posology: Application of the topical composition is effected 4 time per week before sleep, through swiping of the region under the prepuce with a swab containing the composition. The control group uses a commercially available composition named Aldara™, as prescribed by the manufacturer.

Results: Table 6 comprises the results of a 6 weeks treatment with the composition of the present invention on a total of 12 infected patients with warts on the pubic area or under the prepuce. The compiled data consists of the number of recurrent warts that had still developed after a 6 weeks treatment with either the formulation of the cream of the present invention or after treatment with Aldara™. These records represent direct evidence that the composition of the present invention is effective, as the number of warts still present after a 6 weeks long treatment of the intermittent lesion and inflammation being less important with the cream of the composition the present invention, comparable to the commercially available Aldara™. Indeed, 100% of patients treated with the composition of the present invention presented a lower degree of infections after the 6 weeks treatment (1 to 2 recurrent warts). Furthermore, statistical analysis of the results comparing the two treatments with a Student's t-test confirms the significance of this effect: the null hypothesis could not be rejected at a p-value ≦0.01, suggesting that both treatments are as efficient at treating genital warts.

TABLE 6
Results of an 8 weeks treatment with the composition of
the present invention compared to Aldara ™

| Aldara ™ | | Anti-HPV composition | |
|---|---|---|---|
| Subject | Number of Warts | Subject | Number of Warts |

| | | | | | |
|---|---|---|---|---|---|
| 1 | 3 | P | 1 | 4 | UP |
| 2 | 4 | P | 2 | 4 | UP |
| 3 | 5 | UP | 3 | 6 | P |
| 4 | 5 | UP | 4 | 6 | P |
| 5 | 4 | P | 5 | 5 | UP |
| 6 | 4 | P | 6 | 3 | P |

P = Pubic wart;
UP = Under the prepuce

TABLE 7
Results of an 8 weeks treatment with the composition of the present
invention compared to Aldara ™

| TREATMENT (A) | | TREATMENT (B) | |
|---|---|---|---|
| ALDARA ™ | | STANDARDIZED COMPOUND | |
| Number of Warts | | | Number of Warts |
| 2 | N_{A} = 6 | N_{B} = 6 | 1 |
| 1 | Average = 1.7 | Average = 1.5 | 1 |
| 2 | | | 3 |
| 2 | | | 2 |
| 1 | | | 2 |
| 2 | | | 0 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A topical composition for reducing lesion and inflammation of human papillomavirus (HPV) on the skin and mucous membranes comprising an effective amount of at least one component from a) to c):
a) one of ground garlic, garlic powder, garlic oil, minced garlic and greater celandine;
b) a terpene-, phenol- or polyphenol-containing agent; and
c) a sulfurated compound
in association with a pharmaceutically acceptable carrier to form a homogenous topical composition.

2. The topical composition according to claim 1, wherein said garlic powder is present in the composition in a dose consisting of 36 mg per 1 g of a pharmaceutically acceptable carrier.

3. The topical composition according to claim 1, wherein said greater celandine is present in the composition in a dose consisting of 6 drops per 15 g of a pharmaceutically acceptable carrier.

4. The topical composition according to claim 1, wherein said terpene-, phenol- or polyphenol-containing agent is an essential oil selected from the group consisting of thuja oil, eucalyptus oil, birch oil, Atlantic cedar oil, red thyme oil, oregano oil, tarragon oil, red cedar wood oil, basil oil, and combination thereof.

5. The topical composition according to claim 5, wherein said essential oil is present in the composition in a dose consisting of 12 drops per 15 g of a pharmaceutically acceptable carrier.

6. The topical composition according to claim 1, said terpene-, phenol- or polyphenol-containing agent is capsaicin or cayenne pepper.

7. The topical composition according to claim 1, wherein said sulfurated compound is methyl-sulphone-methane or methylsulfonylfluoride.

8. The topical composition according to claim 7, wherein said methyl-sulphone-methane is present in the composition in a dose consisting of 1000 mg per 15 g of a pharmaceutically acceptable carrier and methylsulfonylfluoride is present in the composition in a dose consisting of 12 mg per 15 g of a pharmaceutically acceptable carrier.

9. The topical composition according to claim 1, wherein said pharmaceutically acceptable carrier consists of a lotion or an ointment.

10. A method for reducing lesion and inflammation caused by HPV related infections on skin and mucous membranes of a patient, comprising administering topically a sufficient amount of the topical composition of claim 1 to the skin or mucous membrane of a patient.

11. The method for the treatment according to claim 10, wherein said lesion is a wart.

12. The method for the treatment according to claim 11 wherein said wart an acute or chronic wart.

13. The method for the treatment according to claim 12, wherein said wart is a genital wart.

14. The method for the treatment according to claim 13, wherein said skin and mucous membranes consist of the skin of the penis, vulva (area outside the vagina), anus, scrotum, groin, or thigh and in the linings of the vagina, cervix, or rectum.

15. The method for the treatment according to claim 14, wherein administering topically is effected by swiping the surface of the skin or mucous membrane to be treated with a swab containing the composition in the case of an acute wart.

16. The method for the treatment according to claim 12, wherein administering topically is subcutaneous or transdermal in situ in the case of a chronic wart.

17. The method for the treatment according to claim 16, wherein said transdermal topical administration consists of inserting subcutaneously in situ the composition under the skin or mucous membrane of the subject with a microlet or applying in situ to the skin of the subject a transdermal patch diffusing the composition trough the skin or mucous membrane over an extended period of time.
